(19) United States
(12) Patent Application Publication
(10) Pub. No.: US 2003/0066100 A1
(43) Pub. Date: Apr. 3, 2003

(54) Capacitative calcium entry mechanism in porcine oocytes

(76) Inventors: Zoltan Machaty, Hamilton, NY (US); Jagdeece Ramsoondar, Hamilton, NY (US); Kenneth Bondioli, Hamilton, NY (US)

Correspondence Address: Mark Farber, c/o Alexion Pharmaceuticals, Inc., 352 Knotter Drive, Cheshire, CT, 06410, US

(21) Appl. No.: 10/192,024

(22) Filed: Jul. 9, 2002

Related U.S. Application Data
(60) Provisional application No. 60/304,349, filed on Jul. 9, 2001.

(51) Int. Cl. A01K 67/027; C12N 5/06

(52) U.S. Cl. 800/17; 435/325

(57) ABSTRACT

Mammalian oocytes are contacted with a compound that activates a trp calcium channel. In this manner activation or maturation of the oocyte can be achieved.

### RELATED APPLICATIONS

This application claims priority to U.S. Provisional Application No. 60/304,349 filed Jul. 9, 2001, the disclosure of which is incorporated herein by reference.

### STATEMENT OF GOVERNMENT RIGHTS

At least a portion of the subject matter of this application is based upon work supported by the Cooperative State Research, Education and Extension Service, U.S. Department of Agriculture, under agreement No. 99-35203-7675. Accordingly, the U.S. government may have certain rights herein.

### BACKGROUND

1. Technical Field

This disclosure relates to methods of producing calcium ion influx into mammalian oocytes for activation or maturation of the oocytes, as well as to the activated or mature oocyte.

2. Background of Related Art

Signal transduction at fertilization of mammalian oocytes includes a series of Ca²⁺ transients that are responsible to stimulate meiotic resumption in the oocyte and activate its developmental program The activating signal is believed to be the Ca²⁺ oscillation itself, whose frequency, amplitude, and duration are thought to encode important information that influences subsequent development. The oscillation is generated by the cyclic release of Ca²⁺ from the internal store through specialized Ca²⁺ release channel receptors. The released Ca²⁺ is then re-sequestered into the stores by SERCA (sarcoplasmic endoplasmic reticulum Ca²⁺ ATP-ase) pumps followed by additional release/replenishment cycles. In addition to the release from internal stores it was suggested that a continuous influx of Ca²⁺ through the plasma membrane is necessary to maintain the oscillation. The contribution of Ca²⁺ influx accounts for why the oscillation frequency is sensitive to variations in the level of external Ca²⁺. Ca²⁺ oscillation was inhibited in mouse oocytes by a decrease in the extracellular Ca²⁺ concentration and was totally blocked in the absence of extracellular Ca²⁺ or in the presence of Ca²⁺ influx channel antagonists.

Ca²⁺ signaling in many cell types involves Ca²⁺ oscillations. In excitable cells oscillations arise primarily from the fluctuation in the entry of external Ca²⁺ via voltage-activated calcium channels. On the other hand, agonist stimulation of many non-excitable cells triggers Ca²⁺ release from intracellular stores followed by a Ca²⁺ influx across the plasma membrane. In the latter case, the extracellular Ca²⁺ is probably required to refill the Ca²⁺ pools and this can be attributed to the fact that the majority of Ca²⁺ released from the store is extruded from the cell across the plasma membrane. The Ca²⁺ influx pathway seems to be activated by depletion of the intracellular Ca²⁺ stores and was termed capacitative Ca²⁺ entry. It was postulated that capacitative Ca²⁺ entry plays a role in sustaining Ca²⁺ oscillation that accompanies fertilization in mammalian oocytes and the presence of such a Ca²⁺ entry was observed in mouse oocytes during the Ca²⁺ spikes induced by fertilization or various artificial stimuli.

The capacitative Ca²⁺ entry pathway has not yet been identified. There are a number of channels that can bring Ca²⁺ into cells as a result of store depletion, these channels are generally called store-operated channels. Previously, the transient receptor potential (trp) gene product in Drosophila photoreceptors has been suggested as a promising candidate. The Drosophila trp locus encodes a protein consisting of 1275 amino acids with six putative transmembrane segments; it displays significant similarity to voltage-gated Ca²⁺ channels but lacks the charged amino acids that comprise their voltage sensor. Trp appears to be a key element in the inositol 1,4,5-trisphosphate (InsP₃)-dependent phototransduction process in invertebrates by serving as a Ca²⁺ entry channel. Homologues of trp have been described in several species, however they have never been identified in mammalian oocytes.

It would be advantageous to know definitively whether a capacitative Ca²⁺ entry pathway exists in mammalian oocytes. It would also be advantageous to determine whether a trp homologue exists in mammalian oocytes that can serve as a Ca²⁺ influx channel after store depletion. Manipulating Ca²⁺ influx can be used to achieve activation and/or maturation of mammalian oocytes.

### SUMMARY

It has now been discovered that a trp calcium channel exists in mammalian oocytes. Accordingly, oocyte activation is achieved in accordance with this disclosure by contacting a mammalian oocyte with a compound that activates the trp calcium channel. In this manner an influx of Ca²⁺ is provided, and oocyte activation achieved.

In another aspect, an immature mammalian oocyte is contacted with a compound that activates the trp calcium channel in accordance with this disclosure. In this manner, accelerated and/or improved maturation of the oocyte is provided.

### BRIEF DESCRIPTION OF THE DRAWINGS

FIG. 1 shows Ca²⁺ release in a porcine oocyte induced by 50 μM thapsigargin. The oocyte was held in Ca²⁺-free medium and then thapsigargin (arrow) was added.

FIG. 2 shows capacitative Ca²⁺ entry in porcine oocytes. The intracellular stores were depleted by incubation of the oocytes in Ca²⁺-free medium for 3 h in the presence of 50 μM thapsigargin. Then after a short baseline measurement in Ca²⁺-free medium, Ca²⁺ was added (arrow) to the oocytes (A). The Ca²⁺ entry evoked by store depletion was totally inhibited by 1 mM La³⁺ (B). Each figure represents one oocyte.

FIG. 3 shows divalent cation influx triggered by an InsP₃-induced Ca²⁺ release in porcine oocytes. The injection of 2.5 μM InsP₃ (arrow) triggered an elevation in fluorescence with excitation at 340 nm (lower trace) indicating an increase in [Ca²⁺]ᵢ. Simultaneous measurement at 360 nm (upper trace) revealed only a slight instability in fluorescence; at this wavelength fura-2 fluorescence is insensitive to changes in [Ca²⁺]ᵢ (A). In the presence of 3 mM Mn²⁺ in the external medium, InsP₃ caused a rapid decline in fluorescence (B). This decrease in the fluorescence intensity was due to extracellular Mn²⁺ that entered the oocyte after the InsP₃-induced Ca²⁺ transient and quenched the fluorescence of the intracellular dye at both wavelengths. The arrow marks the addition of Mn²⁺; the Y-axis shows fluorescence in arbitrary units.

FIG. 4 shows Western blot analysis of porcine oocytes injected with mRNA encoding for the Drosophila ctrp-9 protein. The presence of an approximately 150 kDa protein was present in the mRNA-injected oocytes but was absent in the oocytes injected with the carrier medium.

FIG. 5 shows the effect of trp expression on capacitative Ca²⁺ entry. Oocytes were incubated with 50 μM thapsigargin in Ca²⁺-free medium for 2 h. Following a short baseline measurement in Ca²⁺-free medium, Ca²⁺ was added (arrow) to the oocytes. The Ca²⁺ entry in the mRNA-injected oocytes (A) was faster than in the control oocytes (B), due to the higher number of Ca²⁺ entry pathways in the plasma membrane.

FIG. 6 shows RT-PCR products for detecting the presence of a trp homologue in porcine oocytes. RNA was extracted from cells and first strand cDNA was reverse transcribed. PCR was performed for 45 cycles. Lane 1: molecular size marker; lane 2: “no template” control with trp primers; lane 3: ovarian cDNA with trp primers; lanes 4-7: trp cDNA fragment from oocytes; lane 8: “no template” control with β-actin primers; lane 9: ovarian cDNA with β-actin primers; lane 10: oocyte cDNA with β-actin primers.

FIG. 7 shows nucleotide sequence of the PCR product from porcine oocytes together with known human and mouse sequences. The 333 bp fragment amplified from porcine oocytes showed 96.2% identity with the human (Htrp3) and 92.0% identity with the mouse (Mtrp3) sequence.

### DETAILED DESCRIPTION OF PREFERRED EMBODIMENTS

Activation of mammalian oocytes involves exit from meiosis and reentry into the mitotic cell cycle by the secondary oocyte and the formation and migration of pronuclei within the cell. Viable oocytes prepared for maturation and subsequent activation are required for nuclear transfer techniques.

Activation requires cell cycle transitions. The Maturation Promoting Factor complex becomes essential in the understanding of oocyte senescence and age dependent responsiveness to activation. MPF activity is partly a function of calcium (Ca²⁺). A major imbalance in the components of the multi-molecular complex which is required for cell cycle arrest may be responsible for the increasing sensitivity of oocytes to activation stimuli during aging.

It is believed that the most effective activating stimulus would be one that mimicked the response of mammalian oocytes to fertilization. One such response of rabbit oocytes is characterized by repetitive transient elevations in intracellular Ca²⁺ levels followed by rapid return to base line (Fissore and Robl, 1992), which may explain the improved development with activation by multiple electrical pulses.

The present inventors have established for the first time the presence of a trp channel in mammalian oocytes, such as, for example porcine oocytes. Therefore, in accordance with the methods described herein, elevations in intracellular Ca²⁺ levels are achieved by contacting a mammalian oocyte with a compound that activates trp calcium channels. Suitable compounds include, but are not limited to 1,2-dioctanoyl-sn-glycerol (DOG), 1-oleoyl-2-acetyl-sn-glycerol (OAG), 1-stearoyl-2-arachidonyl-glycerol (SAG), Linoleic acid, and Arachidonic acid (AA). While these compounds have been found to activate trp calcium channels, because the existence of trp channels in mammalian oocytes had previously not been known, the present inventors believe themselves to be the first to use these compounds in connection with mammalian oocytes, especially to activate the oocytes.

Although it is contemplated that the procedure described herein may be utilized on a variety of mammals, the procedure will be described with reference to the porcine species. However, the present invention does not restrict the cloning procedure to porcine embryonic cells.

The term “oocyte,” as used herein means an oocyte which develops from an oogonium and, following meiosis, becomes a mature ovum. For purposes of the present disclosure, metaphase II stage oocytes, matured either in vivo or in vitro, are suitable. Mature metaphase II oocytes may be collected surgically from either nonsuperovulated or superovulated gilt or sows 24-48 hours past the onset of estrus or past an injection of human Chorionic Gonadotrophin (hCG) or similar hormone. Alternatively, immature oocytes may be recovered by aspiration from ovarian follicles obtained from slaughtered gilt or sows and then may be matured in vitro in a maturation medium by appropriate hormonal treatment and culturing.

There are a variety of oocyte culture and maintenance media routinely used for the collection and maintenance of oocytes, and specifically porcine oocytes. Examples of known media, which may be used for porcine oocyte culture and maintenance, include Ham's F-10+10% fetal calf serum, Tissue Culture Medium-199 (TCM-199)+10% fetal calf serum, Tyrodes's-Albumin-Lactate-Pyruvate (TALP), Dulbecco's Phosphate Buffered Saline (PBS), Eagle's and Whitten's media. One of the most common media used for the collection and freezing of embryonic cells is TCM-199 and 1 to 20% serum supplement including fetal calf serum, new born serum or steer serum. A suitable maintenance medium includes TCM-199 with Earle's salts, 10% fetal calf serum, 0.2 mM Na-pyruvate and 25 ug/ml gentamicin sulphate. Another maintenance medium is described in U.S. Pat. No. 5,096,822 to Rosenkrans et al., the disclosure of which is incorporated herein by reference. This medium, named CR1, contains the nutritional substances necessary to support an oocyte.

Prior to activation, the cumulus cells can be stripped from the oocytes. Cumulus cells are non-reproductive or somatic cells which surround the oocyte and are believed to provide both protection and nutrients needed to mature the oocyte. The presence of cumulus cells creates a cloud around the oocytes making it very difficult if not impossible to observe oocytes during the maturation period.

Cumulus cells can be stripped from the oocyte using any known technique (e.g., mechanically by pipetting, by vortexing, by ultrasound techniques, etc. or stripped enzymatically by the application of proper enzymes such as trypsin, hyaluronidase or collagenase). The oocytes are then washed according to methods known to the art and moved to a maintenance medium.

The oocyte is then introduced into a medium containing a compound which activates the trp calcium channel thereby causing the introduction of free calcium ion into the oocyte cytoplasm. Calcium is located in the cell membrane, mitochondria, endoplasmic reticula and other parts of the cell as well as externally to the oocyte before being released and introduced as free Ca²⁺ ion into the oocyte cytoplasm. The concentration of the trp calcium channel activating compound in the medium will depend upon a number of factors including, for example, the specific compound used. Typically, the concentration of the trp calcium channel activating compound will be in the range of about 10 nanomolar to about 10 millimolar. The time period for which the oocyte is contacted with the trp calcium channel activating compound will normally be in the range of about 0.5 to about 10 minutes.

Without wishing to be restricted to one source of explanation, it appears that the initial calcium transient appears to be an upstream event which activates a cascade of cellular changes necessary for resumption of meiosis and the cell cycle.

In another aspect, methods for enhancing maturation of a mammalian oocyte is provided herein. In these methods, an immature oocyte is contacted with a compound that activates the trp calcium channel. By conducting maturation in vitro in the presence of a trp calcium channel activating compound, the rate of maturation can be accelerated and the quality of the mature oocyte improved. The compounds and conditions described above for activation of the oocyte are suitable for achieving maturation of an immature mammalian oocyte.

### EXAMPLE

Oocyte Maturation

Experiments were conducted according to institutional Animal Care Use Committee guidelines. All chemicals were obtained from Sigma Chemical Company (St. Louis, Mo.) unless otherwise indicated. Oocyte-cumulus complexes were collected from porcine ovaries and rinsed three times in HEPES-buffered Tyrode's medium containing 0.1% (w/v) polyvinyl alcohol (HEPES-TL-PVA). They were matured in groups of fifty in 500 μl NCSU-23 medium supplemented with 10% porcine follicular fluid, 0.1 mg/ml cysteine, 10 ng/ml EGF, 10 IU/ml eCG and 10 IU/ml hCG. After 22 hours the complexes were transferred into a culture dish containing the same medium without hormones and cultured for an additional 22 h. The cumulus cells were then removed by vigorous pipetting in HEPES-TL-PVA in the presence of 0.3 mg/ml hyaluronidase.

Fluorescent Recordings

The oocytes were loaded with the Ca²⁺ indicator dye fura-2 by being incubated in the presence of 2 μM acetoxymethyl ester form of the dye and 0.02% pluronic F-127 (both from Molecular Probes, Inc., Eugene, Oreg.) for 40-50 minutes. After incubation the oocytes were rinsed, exposed to various treatments and the changes in the intracellular free Ca²⁺ concentration ([Ca²⁺]ᵢ) were followed using a Photoscan-2 photon counting fluorescent microscope system (Nikon Corp., Tokyo, Japan) as described by Macháty et al., Biol Reprod 1997a; 56:921-930. Fluorescence was recorded by calculating the ratio of fura-2 fluorescence at 510 mn excited by UV light alternatively at 340 and 380 nm. Intracellular free Ca²⁺ levels are presented as fluorescent ratio values with ratios of 1.2 and 6.5 representing 65 and 602 nM Ca²⁺, respectively.

Microinjection

To induce the release of Ca²⁺ from the intracellular stores, the second messenger InsP₃ was injected into the oocytes' cytoplasm using a microinjector (Narishige Co. Ltd., Tokyo, Japan). InsP₃ was dissolved in carrier medium consisting of 10 mM Hepes and 100 μM EGTA buffered at pH 7.0. The amount injected was about 40 pl, which is 4% of the total cytoplasmic volume of ˜1000 pl. Microinjection was performed in HEPES-TL-PVA on a heated stage of a Nikon Diaphot inverted microscope (Nikon Corp., Tokyo, Japan).

In vitro Transcription

The plasmid vector pBluescript KS, containing the Drosophila trp cDNA ctrp-9 downstream of the T7 promoter (a generous gift from C. Montell) was transfected into Escherichia coli DH5α cells. Plasmid DNA was isolated and linearized with the restriction endonuclease KpnI (Promega Corp., Madison, Wis.) and mRNA was transcribed from the cDNA with T7 polymerase using the RiboMAX™ Large Scale RNA Production System (Promega), following the manufacturer's recommendations. In order to produce capped RNA transcripts, the reaction was performed in the presence of 3 mM m⁷G(5′)ppp(5′)G (Boehringer-Mannheim Corp., Indianapolis, Ind.). Purified RNA was precipitated with 0.3 M sodium acetate and ethanol. The pellet was resuspended in diethylpyrocarbonate (DEPC)-treated water containing RNasin (1 IU/μl; from Promega) to a final concentration of approximately 800 ng/μl and the samples were stored in 3 μl aliquots at −70° C.

Western Blot

Oocytes injected with ctrp-9 mRNA and control oocytes (injected with DEPC-treated water) were lysed in groups of 20 in 5 μl in denaturing Laemmli sample buffer and boiled for 1 minute. The proteins in the lysate were separated with SDS-PAGE (10% w/v polyacrilamide) and separated proteins were electrophoretically transferred for 2 hours on to polyvinylidene fluoride membranes (Millipore Corp., Bedford, Mass.) for subsequent probing. Immunodetection was achieved by incubating the blots with αzctrp antiserum (an antibody raised in rabbit against the trp protein; a gift from C. Montell) diluted 1:2,000 in PBS with 0.01% Tween-20 and 5% non-fat dry milk. To detect the primary antibody, blots were incubated with horseradish peroxidase-conjugated mouse anti-rabbit IgG antibody diluted 1:5,000 in PBS-0.01% Tween 20-5% non-fat dry milk, washed thoroughly in PBS with 0.01% Tween 20 and exposed to enhanced chemiluminescence reagents for 1 minutes. Subsequently, the blots were exposed to Kodak X-OMAT AR film (Eastman Kodak Co., Rochester, N.Y.).

mRNA Isolation

Poly(A) RNA was extracted from individual oocytes using Hybond-messenger affinity paper (Hybond-mAP; Amersham Pharmacia Biotech, Piscataway, N.J.). Oocytes were incubated with a 3 to 4 mm² piece of Hybond-mAP for 2 hours in guanidium isothiocyanate (GITC) lysis solution (4 M GITC; 0.1 M Tris-HCl, pH 7.4; 1 M beta-mercaptoethanol; all in DEPC-treated water). After incubation, the Hybond-mAP was placed on Whatman filter paper (Fischer Scientific, St. Louis, Mo.) and the aqueous contents of the vials were carefully spotted onto the membrane. The Hybond-mAP was then washed twice in 0.5 M NaCl+0.1 M Tris-HCl, pH 7.4, in DEPC-treated water. This was followed by two additional washes in 0.5 M NaCl in DEPC-treated water and two final rinses in 70% ethanol. The Hybond-mAP was then allowed to air dry for a few minutes and then immediately used for reverse transcription (RT).

Since mammalian trp is expressed at high levels in ovarian tissues, total RNA was isolated from porcine ovaries to be used as a positive control for RT-PCR. Ovaries were flash frozen in liquid nitrogen immediately after removal and stored at −70° C. until processed. For RNA isolation they were removed from the liquid nitrogen, placed into 20 ml lysis buffer (STAT-60; Tel-Test, Inc., Friendswood, Tex.) and homogenized using a rotor-stator homogenizer. An additional 20 ml of lysis buffer was added to the homogenate and it was followed by pipetting 1/10 volume of bromo-chloro-propane to the solution. The mixture was then shaken vigorously for 30 seconds and let sit for 2-3 minutes. Following centrifugation at 10,000 g for 15 minutes, the supernatant was collected into a new tube and the RNA was precipitated by adding an equal volume of ice-cold isopropyl alcohol. The tube was shaken gently, stored at room temperature for 5 minutes and centrifuged at 10,000 g for 15 minutes. The isopropyl alcohol was then poured off, the pellet was washed in ice-cold 80% ethanol and the RNA was aliquoted in DEPC-treated water with 5 μl/ml RNasin. Aliquots were stored at −70° C. until use.

Reverse Transcription

Hybond-mAP with attached RNA was used in the RT reactions, which were carried out under conditions of 42° C. for 45 minutes followed by 95° C. for 5 minutes using a PTC-100 Peltier effect thermocycler with a heated lid (MJ Research, Inc., Watertown, Mass.). The reaction mixtures consisted of the following: 200 IU M-MLV reverse transcriptase, M-MLV reverse transcriptase buffer, 2.5 μM random hexamers, 200 μM each dNTP, and 20 IU RNasin (Promega). Milli-Q water (Millipore) was added to the reaction mixtures to make a final volume of 20 μl.

Total RNA isolated from ovaries was reverse transcribed in a reaction mixture consisting of 200 IU M-MLV reverse transcriptase, M-MLV reverse transcriptase buffer, 200 μM each dNTP, 2.5 μM reverse primer, and 20 IU RNasin. The final volume of 20 μl was achieved by adding Milli-Q water. The RT reaction was carried out by incubating the reaction mixture at 42° C. for 45 minutes followed by a 5 minute incubation at 95° C.

PCR

The primers used to amplify a trp homologue from porcine oocytes were designed based on conserved regions of the murine (Mtrp3) and human (Htrp3) trp homologues. The forward primer was 5′-AAGGACATATTCAAGTTCAT-3′ (SEQ ID NO 1) (bases 2147-2166 of Htrp3 sequence, and the reverse primer was 5′-CCATTCTACATCACTGTCAT-3′ (SEQ ID NO 2) (bases 2460-2479 of Htrp3 sequence). The primers were expected to amplify a 333 bp DNA fragment. As an internal control the following β actin primers were used: forward primer 5′ -GCTGTATTCCCCTCCATCGT-3′ (SEQ ID NO 3), and reverse primer 5′-ACGGTTGGCCTTAGGGTTCA-3′ (SEQ ID NO 4). These primers were able to amplify a 220 bp fragment from porcine cDNA or a 350 bp fragment from genomic DNA. When cDNA from individual oocytes was amplified, the 50 μl PCR reaction mixture contained 5 μl cDNA as a template, 2 mM MgCl₂, 200 μM each dNTP, 2.5 IU Taq polymerase, 1×reaction buffer, 4 nM of each primer, and Milli-Q water. When cDNA from ovaries was used for PCR, the reaction mixture was 25 μl which consisted of 2 μl cDNA, 1 mM MgCl₂, 2.5 IU Taq polymerase, 1×reaction buffer, 1.8 nM forward primer and the appropriate amount of Milli-Q water. The reactions started with 1 cycle of 95° C. for 3 minutes, followed by 45 cycles each of 30 seconds at 95° C. to denature, 30 seconds at 56° C. for annealing and 1 minute at 72° C. for extension, the last cycle was followed by an 8 minutes extension.

Depletion of Ca²⁺ Stores Generates a Ca²⁺ Influx

A Ca²⁺ influx was generated in porcine oocytes by the depletion of the intracellular Ca²⁺ stores. Thapsigargin, a tumor promoting plant sesquiterpene lactone was shown to inhibit the endoplasmic reticulum Ca-ATPases (Ca²⁺ pumps) with little effect on the plasma membrane Ca-ATPase. It is routinely used to drain the intracellular stores of their Ca²⁺ content. Fura-2-loaded oocytes were incubated in Ca²⁺-free HEPES-TL-PVA medium in the presence of 10-50 μM thapsigargin for 3 hours to deplete intracellular Ca²⁺ stores. After washing in Ca²⁺-free medium (to remove thapsigargin and ensure that the intracellular stores remain empty), normal Ca²⁺-containing medium was added to the oocytes and the changes in the [Ca²⁺]ᵢ were measured. Oocytes incubated in Ca²⁺-free HEPES-TL-PVA for 3 hours without thapsigargin were used to show the Ca²⁺ entry under normal conditions, when the intracellular Ca²⁺ stores were full.

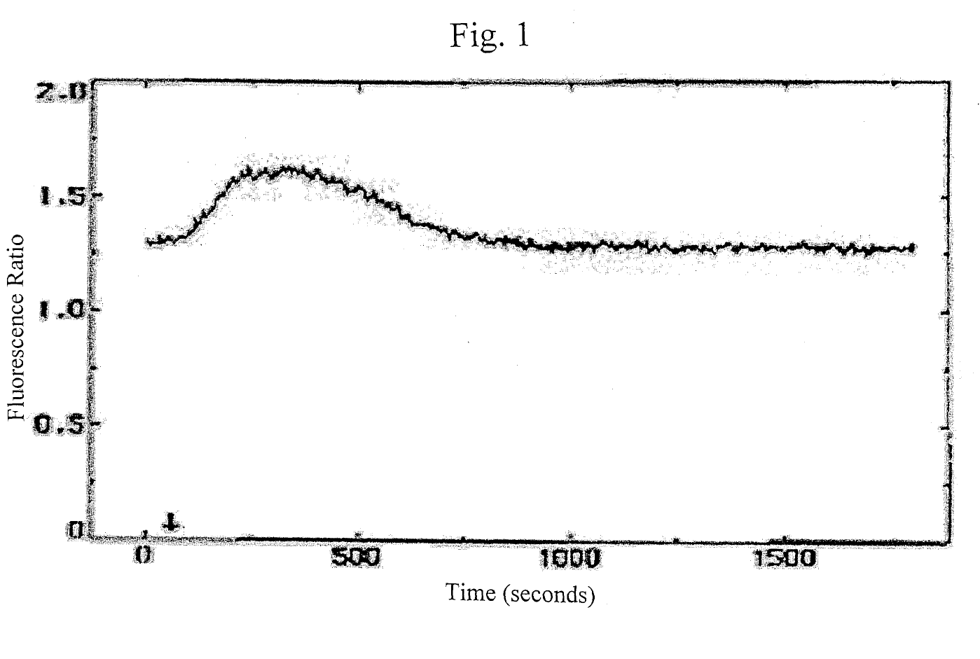

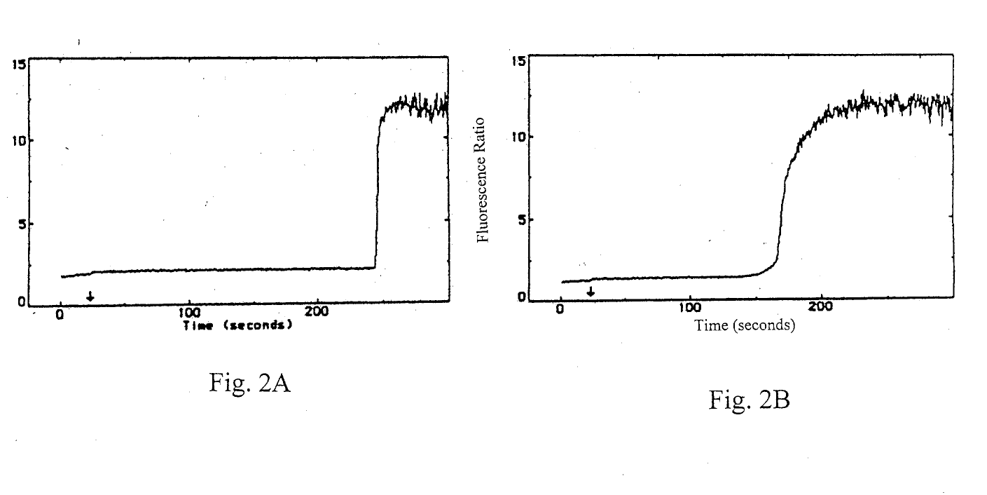

When applied in Ca²⁺-free medium, thapsigargin (10-50 μM) caused the depletion of Ca²⁺ stores and induced an increase in [Ca²⁺]ᵢ in pig oocytes. FIG. 1 shows the response of an oocyte treated with 50 μM thapsigargin, the increase consisted of a slowly rising and falling peak. Concentrations of 10 and 20 μM thapsigargin caused slightly smaller increases in [Ca²⁺]ᵢ. Emptying the intracellular Ca²⁺ stores promoted Ca²⁺ entry after the re-addition of Ca²⁺ in 15 out of 18 oocytes, which was detected as a rise in the [Ca²⁺]ᵢ (FIG. 2A). The increase in [Ca²⁺]ᵢ started 0-300 seconds after adding the HEPES-TL-PVA medium and went on until the end of the measurements, because the blocked pumps could not re-accumulate Ca²⁺ and the empty stores kept sending the activating message to the Ca²⁺ entry pathways infinitely. However, the intracellular Ca²⁺ levels of the control oocytes that were not treated with thapsigargin were not affected by the presence of extracellular Ca²⁺: in these oocytes (12/12) no observable increase in the [Ca²⁺]ᵢ was detected (data not shown).

In Xenopus oocytes the capacitative Ca²⁺ entry pathway could be blocked reversibly by the application of 1 mM Zn²⁺, while in other cells lanthanum (La³⁺) and nickel (Ni²⁺) were reported to block the capacitative Ca²⁺ influx.. In accordance with these reports, the thapsigargin-evoked Ca²⁺ entry in porcine oocytes (11/11) was completely blocked by 1 mM La³⁺ (FIG. 2B). These data show that store depletion triggers Ca²⁺ entry in porcine oocytes, indicating the presence of a capacitative Ca²⁺ entry pathway.

A Ca²⁺ Transient Induces a Divalent Cation Influx

The onset of a divalent cation influx after a Ca²⁺ transient was investigated by using the manganese (Mn²⁺)-quench technique disclosed by Hallam et al., Biochem. J. 1988; 255:179-184. Release of Ca²⁺ from the intracellular stores was stimulated by intracellular injection of approximately 40 pl of 2.5 μM InsP₃, the InsP₃ receptor agonist. As a Ca²⁺ surrogate, Mn²⁺ was added to the external medium. Mn²⁺ was reported to be able to translocate across the plasma membrane, bind fura-2 and quench its fluorescence. This technique enables measurement of divalent cation influx even when Ca²⁺ release from the internal stores is coincident. The entry of Mn²⁺ into the cell was monitored by imaging the resulting quench in fura-2 fluorescence at 510 nm excited alternatively at 340 and 360 nm. While the signal resulting from the 340 nm excitation is [Ca²⁺]ᵢ sensitive, at 360 nm fura-2 fluorescence is independent of [Ca²⁺]ᵢ and any decrease in fluorescence is due only to Mn²⁺ entry.

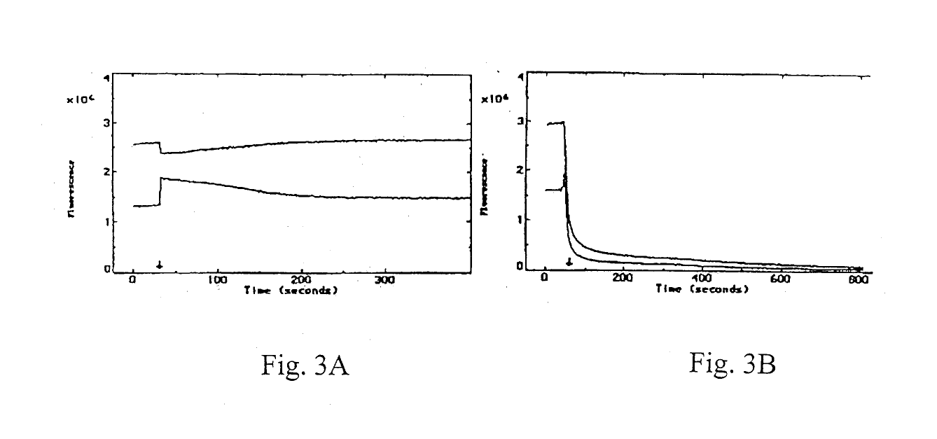

InsP₃ induced a transient elevation in fluorescence with excitation at 340 nm in 16 out of 16 oocytes, indicating an increase in the ([Ca²⁺]₁). After the Ca²⁺ transient, the signal returned to the resting value. Simultaneous measurement at 360 nm revealed only a slight instability in fluorescence (FIG. 3A). At this wavelength, fura-2 fluorescence is insensitive to changes in [Ca²⁺]ᵢ. When the oocytes were microinjected with InsP₃ in the presence of 3 mM Mn²⁺ in the external medium (or alternatively, Mn²⁺ was added subsequent to microinjection) there was a rapid decline in fluorescence well below the basal value (14/14 oocytes). This decrease in the fluorescence intensity was due to extracellular Mn²⁺ that entered the oocyte after the InsP₃-induced Ca²⁺ transient and quenched the fluorescence of the intracellular dye (FIG. 3B). The basal rate of fluorescence quenching due to Mn²⁺ translocation across the plasma membrane in the control noninjected oocytes was considerably less.

La³⁺, the inhibitor of Ca²⁺ entry channels, totally blocked the cation influx and hence the decline in fluorescence at both wavelengths. When InsP₃ was microinjected in the presence of 1 mM La³⁺, the fluorescence intensities stayed near the resting values, even after the addition of Mn²⁺ in all cases (7/7; data not shown). These results strengthen the idea that a capacitative Ca²⁺ entry mechanism exists in porcine oocytes, i.e. the discharge of Ca²⁺ from intracellular stores stimulates an inward Ca²⁺ current that might play a role in refilling the stores.

Heterologous Expression of trp Channels Increased Ca²⁺ Influx

The Drosophila trp protein was expressed in porcine oocytes by injecting approximately 32 pg mRNA made by in vitro transcription of the cDNA and allowing 15 hours for translation. Control oocytes were injected with the carrier medium (DEPC-treated water). The injected oocytes were stained with the Ca²⁺ indicator dye fura-2 AM and incubated in Ca²⁺-free HEPES-TL-PVA with 50 μM thapsigargin for 2 h. Since a 3 hour long thapsigargin-incubation stimulated very distinct capacitative Ca²⁺ entry, probably due to complete store depletion, the incubation time in this experiment was reduced to 2 hours so that any difference between injected and non-injected oocytes would be more apparent. The baseline fluorescence of the oocytes was then recorded in Ca²⁺-free HEPES-TL-PVA, and changes in [Ca²⁺]ᵢ were measured for 20-30 minutes after the addition of Ca²⁺-containing medium.

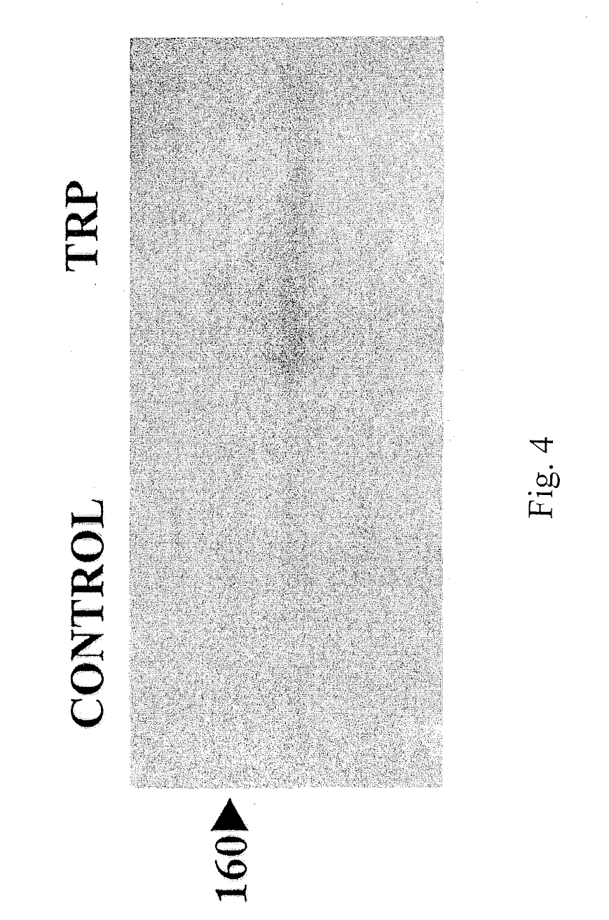

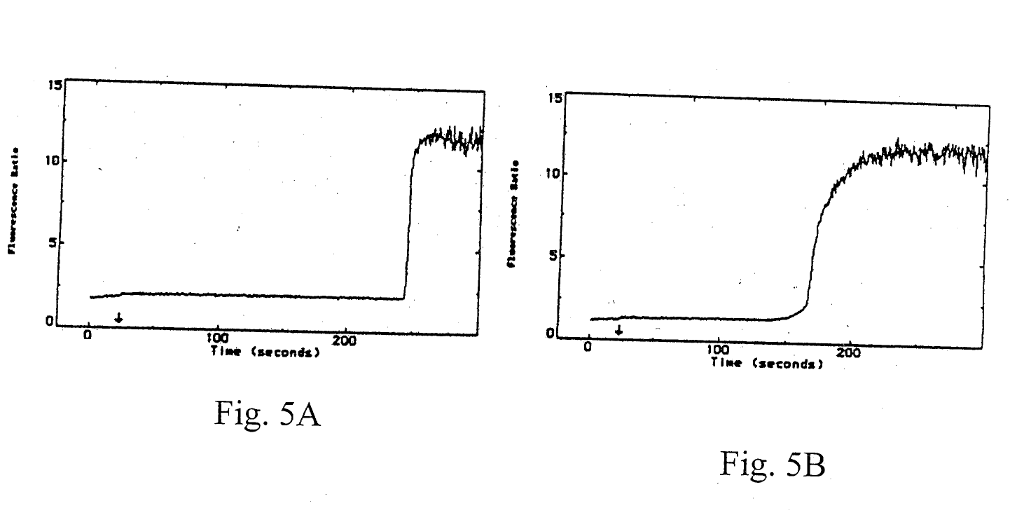

The Drosophila trp protein was expressed in porcine oocytes by injecting approximately 32 pg mRNA encoding the trp channel. The existence of an approximately 150 kDa protein was demonstrated in the mRNA-injected oocytes by western blot analysis using αzctrp, an antiserum raised against the Drosophila ctrp-9 protein. In the control oocytes this protein was not present (FIG. 4). Application of external Ca²⁺ after thapsigargin treatment to carrier medium-injected oocytes induced a Ca²⁺ influx indicating the presence of the endogenous capacitative Ca²⁺ entry mechanism. However, the increase in the [Ca²⁺]ᵢ caused by Ca²⁺ entry occurred faster in oocytes expressing Drosophila trp: the time required for the baseline Ca²⁺ to reach its maximum value and begin to oscillate was significantly shorter in the mRNA-injected oocytes than in the carrier medium-injected oocytes (8.0±2.3 secondsvs. 27.0±2.8 s; P<0.001; FIGS. 5A, B). The Ca²⁺ entry-evoked [Ca²⁺]ᵢ increase was completely blocked by 1 mM La³⁺ (data not shown). These findings suggest that trp homologues expressed in porcine oocytes may function as Ca²⁺ entry channels.

Porcine Oocytes Contain trp mRNA

The existence of RNAs in the porcine oocyte that are homologous with trp was confirmed as follows. Poly(A) RNA was isolated from the oocytes and cDNA was prepared by reverse transcription PCR (RT-PCR). The primers used for the PCR were designed as described above. The PCR products were electrophoresed on a 1.8% agarose gel, isolated and cloned into the plasmid vector pCR2.1 (Invitrogen; Carlsbad, Calif.). Plasmids containing inserts of the correct size were sequenced by MWG Biotech, Inc. (High Point, N.C.). Sequencing of the PCR product was expected to show whether porcine oocytes contain a mammalian homologue of trp, and serve to compare the homology between human (and mouse) trp and the trp found in porcine oocytes.

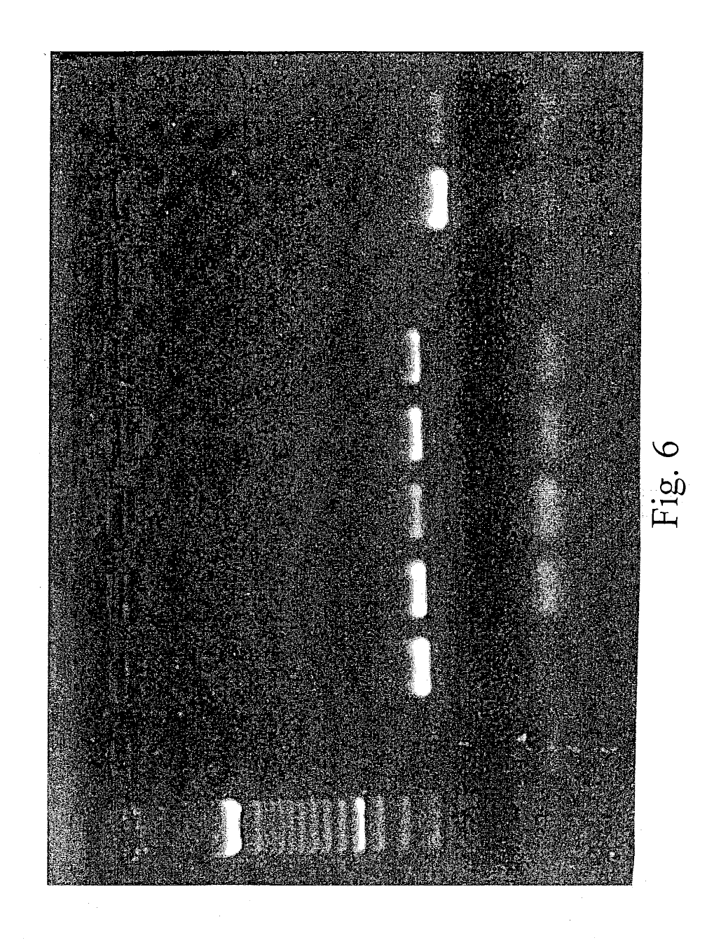

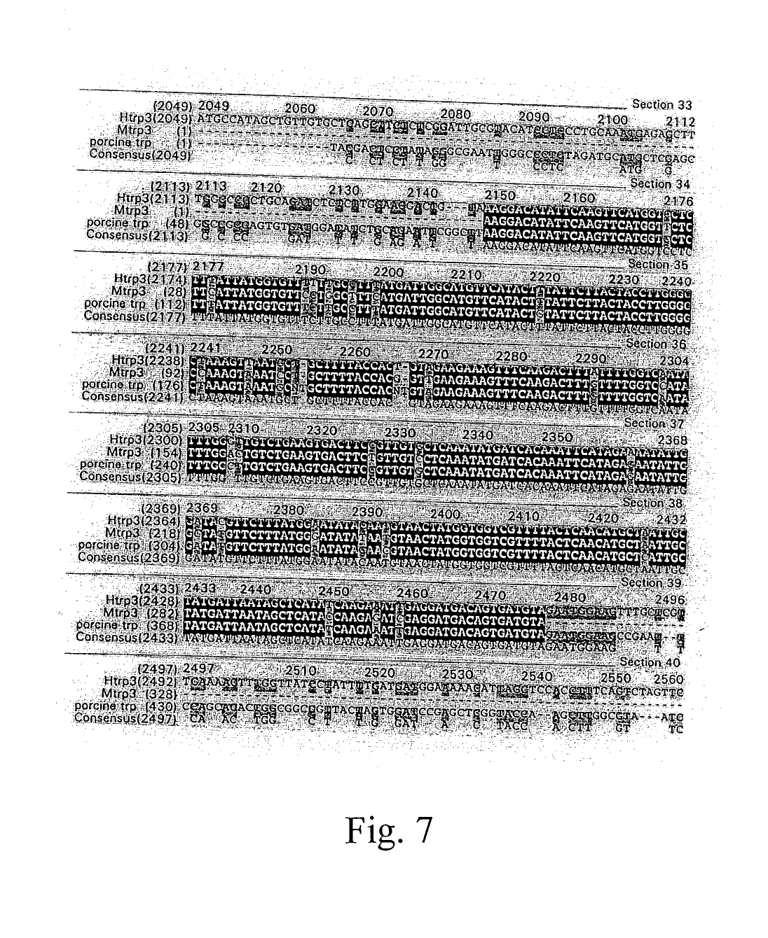

PCR amplification revealed the expected 333 bp band from both oocyte and ovary cDNA (FIG. 6). Sequencing of the PCR product showed that the band amplified from porcine oocyte cDNA corresponded with the murine (Mtrp3) and human (Htrp3) trp sequences and showed 92.0% identity with Mtrp3 and 96.2% identity with Htrp3 (FIG. 7; GenBank accession number: AF420483). This indicates that porcine oocytes express a trp homologue.

The experiments described above clearly indicate the presence of a capacitative Ca²⁺ entry mechanism in porcine oocytes. First, it was demonstrated with the use of thapsigargin, the plant sesquiterpene lactone. Thapsigargin induces a passive depletion of intracellular Ca²⁺ stores by inhibiting the SERCA pumps. In porcine oocytes it also induced a small Ca²⁺ transient in the absence of extracellular Ca²⁺ indicating the depletion of the Ca²⁺ pools and, consistent with the capacitative entry model, it activated a substantial Ca²⁺ influx after the re-addition of Ca²⁺. The thapsigargin concentration used in these experiments is higher than that normally used in somatic cells, it is comparable to the concentrations reported in a study in mouse oocytes. Since thapsigargin acts directly on the SERCA pumps without generating any Ca²⁺-releasing second messengers, such a result indicates that depletion of Ca²⁺ stores provides sufficient signal for the activation of Ca²⁺ entry. This has been confirmed in a large number of cells where the Ca²⁺ influx pathways also remained activated as long as the intracellular pools were not permitted to refill. Originally it was postulated that Ca²⁺ influx pathways would take Ca²⁺ directly into the Ca²⁺ stores without elevating free Ca²⁺ levels in the cytosol. However, later it was shown that repletion Ca²⁺ first enters the cytoplasm (thus the entry is associated with an increase in [Ca²⁺]ᵢ) from which SERCA pumps transport it into the endoplasmic reticulum. Our results are also in accordance with the findings in mouse oocytes where the addition of Ca²⁺ to the oocytes after the thapsigargin-induced Ca²⁺ transient was able to induce a Ca²⁺ influx.

The presence of capacitative Ca²⁺ entry was also demonstrated after intracellular injection of the Ca²⁺ signaling molecule, InsP₃. Normally, InsP₃ is generated by the hydrolysis of membrane phospholipids, it then binds to its receptor located in the endoplasmic reticulum which results in a rapid release of Ca²⁺ to the cytoplasm. The Ca²⁺ release induced by InsP₃ stimulated an immediate divalent cation entry as shown by the Mn²⁺ quench technique. Since InsP₃ was implicated in intracellular Ca²⁺ release during fertilization, the Mn²⁺ influx activated by the InsP₃-induced Ca²⁺ release indicates that capacitative Ca²⁺ entry can be stimulated with physiological second messengers in porcine oocytes. This was clearly demonstrated in mouse oocytes where the stimulation of cation influx was associated with the fertilization Ca²⁺ spikes.

The identity of the capacitative Ca²⁺ entry channels is not known. There are various pathways by which extracellular Ca²⁺ can enter the cell including channels operated by voltage, by receptors, or by second messengers. To distinguish it from other Ca²⁺ entry channels, the term Ca²⁺ release-activated Ca²⁺ current (I_{CRAC}) was used to refer to the current flowing through the capacitative Ca²⁺ entry channels. I_{CRAC} is probably the most meticulously characterized Ca²⁺ influx current but molecularly the entry channel has not yet been classified. A very promising candidate for a CRAC-like protein has been the mammalian homologue of the Drosophila protein trp. During visual signal transduction in invertebrates, light induces the release of Ca²⁺ from intracellular stores followed by photoreceptor depolarization and the development of the so-called receptor potential. It is also followed by the activation of two membrane channels, trp and trp-like (trp1), which in turn admit Ca²⁺ and other cations into the cell and depolarize it. In wild type flies, if light persists, the receptor potential is sustained by this Ca²⁺ influx. In trp-deficient flies photostimulation causes only a transient receptor potential (trp) because the photoreceptors are unable to sustain an influx of Ca²⁺ through the membrane channels.

After the cloning of Drosophila trp gene, the presence of trp homologues were identified in several species. Its presence was also shown in porcine aortic endothelial cells. Trp was first suggested to be a capacitative Ca²⁺ entry channel by Hardie et al., Trends Neurosci. 1993; 16:371-376. Expression of trp in insect Sf9 cells resulted in a depletion-activated inward current. When expressed in Xenopus oocytes, trp enhanced Ca²⁺ influx after thapsigargin treatment. Moreover, the rat trp homologue, when expressed in Xenopus oocytes, also stimulated increased Ca²⁺ conductance, and the human trp homologue expressed in a mammalian cell line enhanced store-operated Ca²⁺ entry. The results of the experiments described herein are consistent with these results. In trp-expressing porcine oocytes the increase of the Ca²⁺ concentration due to Ca²⁺ influx reached maximum levels significantly faster than in control oocytes. This is probably due to the increased number of Ca²⁺ entry channels in the plasma membrane.

To date, seven mammalian trp homologues have been identified. (See, Tomita et al., Neurosci Lett 1998; 248:195-198. The present inventors are believed to be the first to discover the existence of a trp homologue in a mammalian oocyte. The cDNA fragment from porcine oocytes showed 92.0% identity with mouse and 96.2% identity with the human trp sequences. Electrophysiological studies on single channel activity are needed to verify whether this trp channel can serve as capacitative Ca²⁺ entry pathway after depletion of intracellular stores, or the Ca²⁺ influx through these channels simply represents additional Ca²⁺ entry. Although several studies showed that trp1, trp4 and trp5 may function as store-operated channels, others demonstrated that mammalian trp channels are not activated by store depletion, at least when heterologously expressed. Moreover, data suggest that trp3 functions as a Ca²⁺-activated nonselective cation channel and the thapsigargin-induced Ca²⁺ entry in trp3-expressing cells is due to activation of this channel by Ca²⁺ entering through the endogenous capacitative entry pathway. Similarly, trp6 transfected COS.M6 cells showed augmented Ca²⁺ entry only after surface receptor activation, and not after store depletion by thapsigargin. Using cell-attached patch recordings to monitor trp1 single channel activity it was demonstrated that thapsigargin induced an increase in trp1 activity in the presence of extracellular Ca²⁺ when expressed in Sf9 cells. However, the increase in trp1 activity was blocked by low-micromolar concentrations of La³⁺ that previously completely inhibited endogenous capacitative Ca²⁺ entry but had no effect on cation flux via trp1, suggesting that trp1 channel activity requires Ca²⁺ entry via the endogenous capacitative Ca²⁺ entry pathway. Heterologous expression of trp1 was also shown to give rise to cation currents that are not activated by the depletion of internal stores but are stimulated following activation of membrane receptors linked to phosphoinositide turnover. The trp channel and CRAC, the typical capacitative Ca²⁺ entry channel, also have different permeability properties: trp has a higher conductance and it is also much less specific than the CRAC channel.

In summary, porcine oocytes were shown to have a capacitative Ca²⁺ entry mechanism that is activated after depletion of intracellular stores by SERCA pump inhibition or following a Ca²⁺ transient induced by the second messenger InsP₃. Heterologous expression of the Drosophila trp protein in these oocytes increases Ca²⁺ influx following store depletion. Porcine oocytes also contain mRNA homologous with mouse and human trp molecules indicating that the oocytes express a trp homologue.

It will be understood that various modifications may be made to the embodiments described herein. Therefore, the above description should not be construed as limiting, but merely as exemplifications of preferred embodiments. Those skilled in the art will envision other modifications within the scope and spirit of the claims appended hereto.

We claim:

1. A method comprising:
providing a mammalian oocyte; and
contacting the mammalian oocyte with a medium containing a compound that activates a trp calcium channel.

2. A method as in claim 1 wherein the compound that activates a trp calcium channel is selected from the group consisting of 1,2-dioctanoyl-sn-glycerol (DOG), 1-oleoyl-2-acetyl-sn-glycerol, 1-stearoyl-2-arachidonyl-glycerol, linoleic acid, and arachidonic acid.

3. A method as in claim 1 wherein the compound that activates a trp calcium channel is present in the medium at a concentration in the range of 10 nanomolar and 10 millimolar.

4. A method as in claim 1 wherein the oocyte is contacted with the medium for a period of time in the range of 0.5 to 10 minutes.

5. A method as in claim 1 wherein the mammalian oocyte is a porcine oocyte.

6. A method as in claim 1 wherein the oocyte has been matured in vivo.

7. A method as in claim 1 wherein the oocyte has been matured in vitro.

8. A method comprising:
providing an immature mammalian oocyte; and
contacting the immature mammalian oocyte with a medium containing a compound that activates a trp calcium channel.

9. A method as in claim 8 wherein the compound that activates a trp calcium channel is selected from the group consisting of 1,2-dioctanoyl-sn-glycerol (DOG), 1-oleoyl-2-acetyl-sn-glycerol, 1-stearoyl-2-arachidonyl-glycerol, linoleic acid, and arachidonic acid.

10. A method as in claim 8 wherein the compound that activates a trp calcium channel is present in the medium at a concentration in the range of 10 nanomolar and 10 millimolar.

11. A method as in claim 8 wherein the oocyte is contacted with the medium for a period of time in the range of 0.5 to 10 minutes.

12. A method as in claim 8 wherein the mammalian oocyte is a porcine oocyte.

13. A method as in claim 9 wherein the mammalion oocyte is a porcine oocyte.

14. A method as in claim 2 wherein the mammalion oocyte is a porcine oocyte.

15. A composition comprising a mammalian oocyte and a compound that activates a trp calcium channel.

16. A composition as in claim 15 wherein the compound that activates a trp calcium channel is present in the medium at a concentration in the range of 10 nanomolar and 10 millimolar.

17. A composition as in claim 15 wherein the mammalian oocyte is a porcine oocyte.

18. A composition as in claim 15 wherein the mammalian oocyte is an immature oocyte.

19. An activated mammalian oocyte produced by the method of claim 1.

20. A mature mammalian oocyte produced by the method of claim 8.
